(19) United States
(12) Patent Application Publication
(10) Pub. No.: US 2014/0107699 A1
(43) Pub. Date: Apr. 17, 2014

(54) ACTIVE LOADED FIXATION DEVICES

(71) Applicant: SMITH & NEPHEW, INC., MEMPHIS, TN (US)

(72) Inventor: Andrew Fraser Thompson, York (GB)

(73) Assignee: SMITH & NEPHEW, INC., MEMPHIS, TN (US)

(21) Appl. No.: 14/052,143

(22) Filed: Oct. 11, 2013

(30) Foreign Application Priority Data
Oct. 11, 2012 (GB) 1218265.5

(51) Int. Cl. C12N 5/00; A61L 17/00

(52) U.S. Cl. CPC C12N 5/0081; A61L 17/005
USPC 606/231; 435/2

(57) ABSTRACT

The present disclosure relates to methods for producing, including intra-operatively obtaining, a fixation device, and fixation devices produced thereby, containing an autologous cell concentrate from a physiological solution, wherein the cell concentrate is isolated without the need for centrifugation.

### CROSS REFERENCE TO RELATED APPLICATION

This application claims benefit to GB 1218265.5, filed 11 Oct. 2012, the entire contents of which are hereby incorporated by reference.

### BACKGROUND

The present disclosure relates generally to fixation devices loaded with bioactive materials. More specifically, the present disclosure relates to fixation devices, such as sutures, loaded with bioactive materials, such as autologous cell concentrates, and methods for making such devices.

It is a general aim of medical practitioners to promote healing at the site of treatment following surgery. A consideration in the promotion of tissue repair is the extent to which reparative cells and other biological factors can permeate through to the tissue in question. This, in turn, is dependent upon the blood supply to the site and, more specifically, the extent to which blood vessels can form in and around the site.

The use of agents which promote tissue growth is generally known. It is also known to coat such agents onto fixation devices such as sutures, surgical staples, screws and nails to enhance the healing process in tissue which is slow to heal.

Active agents such as growth factors have been shown to help promote tissue healing. However, commercially available examples tend to be expensive. Therefore, autologous agents concentrated from a patient's own body fluids, such as blood or aspirate from bone marrow, may represent a more appropriate, therapeutically efficacious and cost effective alternative.

Traditional autologous components can be extracted from body fluids and concentrated through the use of centrifuges. The isolated material can then be loaded onto a medical device, such as suture, by transferring to appropriate containers. This process is inefficient and requires the use of multiple sterile vessels for each process involved—from extraction of body fluids from the patient, isolation of the active material, its concentration, and subsequent loading of the medical device. This is not only a costly and time-consuming process, but the multiple transfers increase the possibility of a contamination of the sample occurring.

In one example, U.S. Pat. No. 7,837,708, describes a suture combined intraoperatively with autogenous blood components. According to the described process, blood obtained from a patient is separated, using a centrifuge, to retrieve components such as autogenous growth factors and obtain an autogenous blood suspension. The autogenous blood suspension is added to a sterile container containing at least one strand of suture, and the suture subsequently absorbs biologic components of the autogenous blood suspension to produce an enhanced suture. Surgical repairs using the enhanced suture may be conducted by suturing a tear to itself or to bone, for example. Post-operatively, the biologic components are thought to leach from the suture to accelerate healing of the repair.

In addition, containers for loading biologically active materials on suture have been described. For example, U.S. Patent Application Publication No. 20070170080 discloses a package containing a sealable pouch having suture contained therein. The pouch includes a sealed port to allow an active agent to be introduced into the sealable pouch to coat the suture. Further, for example, U.S. Patent Application Publication No. 20080171972 describes a container for receiving suture having a region configured for storing at least one biologically active agent, and a port for introducing suture into the container, particularly the area configured for storing the agent.

As generally disclosed in the art, each of the above described systems require the use of a centrifuge for isolating the biologically active materials. The use of centrifuges increases the amount of time required in the operating theatre and also involves multiple transfers of the biologically active materials, which in itself is time-consuming and also increases the risk of contamination. There process is also relatively expensive since it requires one or more centrifuges and various other disposables required in the isolation and centrifugation procedures.

A more convenient approach could have considerable advantages over currently disclosed systems. For example, the preparation times and associated costs could be significantly reduced. This could help address the lack of presently disclosed relatively simple, low cost devices and methods for loading autologous agents on to medical devices, such as sutures, and devices so made. This includes, for example, the production of loaded fixation devices intraoperatively, including, for example, in a few steps, such as one or two steps.

The present invention seeks to address at least some of the apparently undisclosed systems, methods, and devices, for example, for conveniently, efficiently, and economically preparing fixation devices having, for example, enhanced properties due to the presence of autologous cell concentrates within the devices.

### SUMMARY

The present disclosure relates to methods for producing, including intra-operatively obtaining, a fixation device containing an autologous cell concentrate from a physiological solution, wherein the cell concentrate is isolated without the need for centrifugation, and fixation devices produced thereby.

The present disclosure relates to methods for producing fixation devices having autologous cell concentrates from physiological solutions including: providing a physiological solution not previously subjected to centrifugation; subjecting the physiological solution to at least one filter to produce at least a first filter retentate and a first filter permeate solution, the first filter retentate comprising platelets, nucleated cells, or both, per unit volume greater than in the physiological solution, and wherein the first permeate solution comprises plasma and red blood cells; removing the first filter retentate from the filter to provide an autologous cell concentrate; and subjecting a fixation device to at least the first autologous cell concentrate.

The present disclosure further relates to the method described above, wherein the fixation device is prepared intra-operatively.

The present disclosure further relates to the method described above, wherein the first filter is a nucleated cell filtration device. In some embodiments, the first filter is a leukocyte reduction filter.

The present disclosure further relates to the method described above, further wherein the first retentate is subjected to a second filtration.

The present disclosure further relates to the method described above, wherein the second filtration utilizes a second filter comprising a hollow fiber filter.

The present disclosure further relates to the method described above, the first and/or second filters comprise a pore size between about 0.05 μm and about 5 μm.

The present disclosure further relates to the method described above wherein the first and/or second filters comprise a pore size between about 0.2 μm and about 0.5 μm.

The present disclosure further relates to the method described above wherein an additional solution containing bone marrow aspirate, blood, or a mixture thereof is combined with the physiological solution.

The present disclosure further relates to the method described above wherein the nucleated cells include at least one cell from the group of stem cells, connective tissue progenitor cells, osteoprogenitor cells, or chondroprogenitor cells.

The present disclosure further relates to the method described above wherein the stem cells are mesenchymal stem cells, hematopoietic stem cells, or both.

The present disclosure further relates to the method described above wherein the fixation device is at least one of the group of sutures, tissue anchors, or bone anchors.

The present disclosure further relates to the method described above wherein the fixation device is a suture.

The present disclosure further relates to the method described above wherein the physiological solution is provided by aspirating bone marrow from an individual into a first syringe to produce a bone marrow aspirate.

The present disclosure further relates to the method described above wherein the first syringe contains an anti-coagulant, an isotonic solution, or both.

The present disclosure further relates to the method described above wherein the filtration and intra-operative loading is carried out in a single step.

The present disclosure also relates to fixation devices having at least one component that is substantially coated with an autologous cell concentrate.

The present disclosure further relates to fixation devices having at least one component that is substantially coated with an autologous cell concentrate wherein the autologous cell concentrate contains substantially platelets, nucleated cells, or both, and substantially no plasma or red blood cells.

The present disclosure further relates to fixation devices having at least one component that is substantially coated with an autologous cell concentrate wherein the autologous cell concentrate is prepared by subjecting a physiological solution to a filter to produce a filter retentate and a permeate solution, the filter retentate having platelets, nucleated cells, or both per unit volume greater than in the physiological solution, and wherein the permeate solution has plasma and red blood cells.

The present disclosure further relates to fixation devices having at least one component that is substantially coated with an autologous cell concentrate wherein the fixation device is prepared intra-operatively.

The present disclosure further relates to fixation devices having at least one component that is substantially coated with an autologous cell concentrate wherein the autologous cell concentrate is prepared by subjecting a physiological solution to a nucleated cell filtration device.

The present disclosure further relates to fixation devices having at least one component that is substantially coated with an autologous cell concentrate wherein the autologous cell concentrate is prepared by subjecting a physiological solution to a filter having a pore size between about 0.05 μm and about 5 μm.

The present disclosure further relates to fixation devices having at least one component that is substantially coated with an autologous cell concentrate wherein the autologous cell concentrate is prepared by subjecting a physiological solution to a filter having a pore size between about 0.2 μm and about 0.5 μm.

The present disclosure further relates to fixation devices having at least one component that is substantially coated with an autologous cell concentrate wherein the autologous cell concentrate contains at least bone marrow aspirate, blood, or a mixture thereof.

The present disclosure further relates to fixation devices having at least one component that is substantially coated with an autologous cell concentrate wherein the autologous cell concentrate contains at least nucleated cells containing at least one cell from the group of stem cells, connective tissue progenitor cells, osteoprogenitor cells, chondroprogenitor cells, or combinations thereof.

The present disclosure further relates to fixation devices having at least one component that is substantially coated with an autologous cell concentrate wherein the autologous cell concentrate contains at least stem cells are mesenchymal stem cells, hematopoietic stem cells, or both.

The present disclosure further relates to fixation devices having at least one component that is substantially coated with an autologous cell concentrate wherein the autologous cell concentrate contains at least bone marrow aspirate, blood, or a mixture thereof.

The present disclosure further relates to fixation devices having at least one component that is substantially coated with an autologous cell concentrate wherein the fixation device is at least one of the group sutures, tissue anchors, bone anchors, or combinations thereof.

The present disclosure further relates to fixation devices having at least one component that is substantially coated with an autologous cell concentrate wherein the fixation device is a suture.

The present disclosure further relates to fixation devices having at least one component that is substantially coated with an autologous cell concentrate wherein the at least one component of the fixation device is at least one strand of the suture.

The present invention also relates to an autologous cell concentrate coated suture wherein at least one strand of a suture is substantially coated with an autologous cell concentrate containing at least one of platelets, nucleated cells, or both.

The present invention further relates to an autologous cell concentrate coated suture wherein at least one strand of a suture is substantially coated with an autologous cell concentrate containing at least one of nucleated cells of from the group containing stem cells, connective tissue progenitor cells, osteoprogenitor cells, chondroprogenitor cells, or combinations thereof.

The present invention further relates to an autologous cell concentrate coated suture wherein at least one strand of a suture is substantially coated with an autologous cell concentrate containing at least stem cells.

The present invention further relates to an autologous cell concentrate coated suture wherein at least one strand of a suture is substantially coated with an autologous cell concentrate containing at least one of osteoprogenitor cells, chondroprogenitor cells, or both.

### BRIEF DESCRIPTION OF THE DRAWINGS

So that the above recited features and advantages of the disclosure can be understood in detail, a more particular description of the disclosure, briefly summarized above, may be had by reference to the embodiments thereof that are illustrated in the appended drawings. It is to be noted, however, that the appended drawings illustrate only typical embodiments of this disclosure and are, therefore, not to be considered limiting of its scope. The figures are not necessarily to scale, and certain features and certain views of the figures may be shown exaggerated in scale or in schematic in the interest of clarity and conciseness.

FIGS. 1A-B are embodiments of loading devices for a fixation device, in accordance with a system of the invention;

FIG. 2 is an embodiment of an alternative loading device for a fixation device, in accordance with a system of the invention;

FIG. 3A-B are side and top-plan views of a further alternative loading device for a fixation device, in accordance with a system of the invention; and

FIGS. 4A-B are embodiments of a system in accordance with the invention, which system includes the loading device of FIGS. 1A-B.

### DETAILED DESCRIPTION

The description that follows includes exemplary apparatuses, methods, techniques, and instruction sequences that embody techniques of the inventive subject matter. However, it is understood that the described embodiments may be practiced without these specific details.

Plural instances may be provided for components, operations or structures described herein as a single instance. In general, structures and functionality presented as separate components in the exemplary configurations may be implemented as a combined structure or component. Similarly, structures and functionality presented as a single component may be implemented as separate components. These and other variations, modifications, additions, and improvements may fall within the scope of the inventive subject matter.

Various suture loading devices may be used. These comprise of three main components; a holding body to contain the device (which is preferably supplied sterile with the device in situ), a connector such as luer lock to connect the device to a loading device, such as a syringe, or directly to the filter mechanism, and an orifice for removing medical device for use by the health professional.

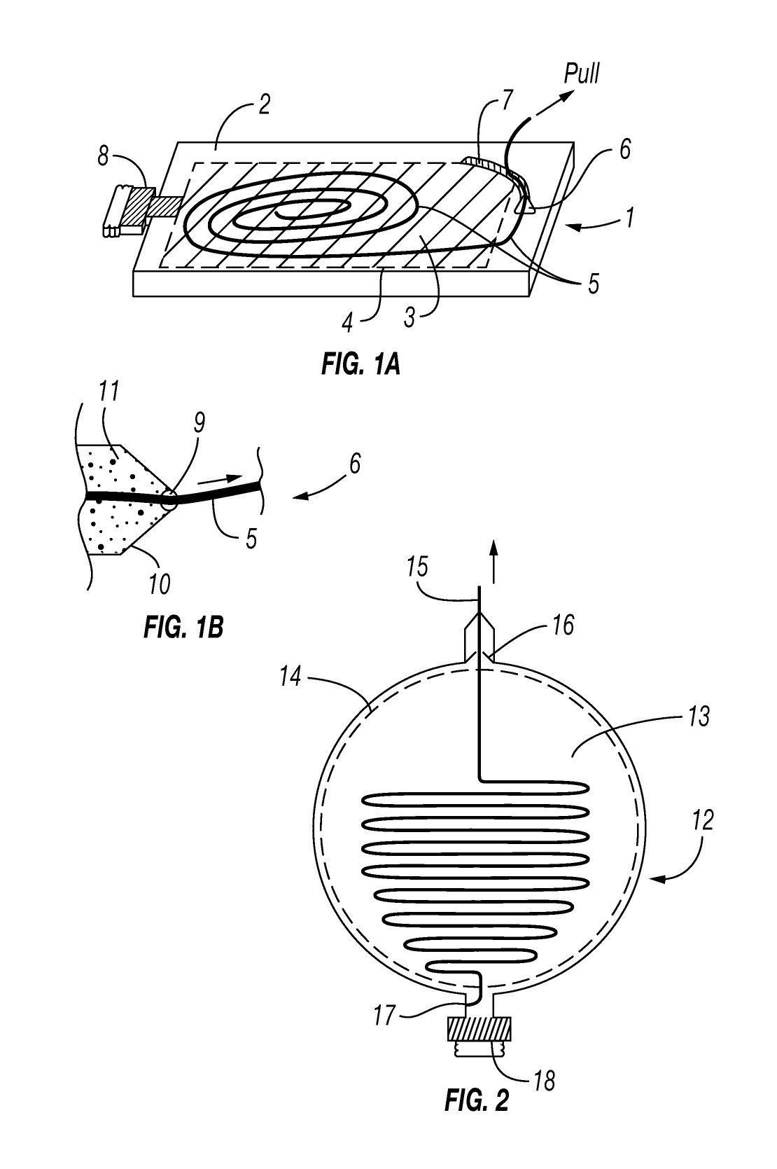

In an embodiment (FIG. 1.a) the medical device loading device 1 consists of a rectangular package 2. The package 2 can be made out of a solid or malleable material such as a polymer material, and its purpose is to protect the inside of the loading device once it contains an active loading fluid. Internally the loading device consists of a reservoir 3 which contains the medical device which is preferably a suture material 5. The reservoir is impermeable and it acts to contain a fluid, preferably a blood based product in the package 2 with the suture device 5. The internal reservoir 3 is attached to a connector port 8 such as a luer lock which is a docking point to attach the device 1 directly to a filter based blood concentrator or active loaded syringe. The rectangular package 2 also comprises an exit point for the medical device which is further illustrated in (FIG. 1.b). The cone shaped exit point 6 has an exit hole 9 with sloped side walls 10. The exit hole 9 forms a close fit with the suture material 5. Upon removal of the suture material 5 the autologous component 11 is kept within the conical structure 10. This structure 6 has the advantage of keeping the autologous material 11 behind the suture when it removed which ensure efficient loading and ease of use for the health professional.

Alternatively the medical device can be contained in a thick walled bag construct (FIG. 2). The medical device, preferably a suture material 15 is supplied within a bag loading device 12. The bag loading device can be manufactured out of a multitude a materials but preferably a thick wall polymer material. The bag loading device 12 can be manufactured in two half's. Once the suture material 15 has been positioned within the bag device the two halves can be placed together and sealed 14 using techniques such as hot crimping. This leaves an open area 13 which contains the suture material 15 and allows for loading of autologous factors or other active agents. The suture material is secured 17 weakly at the bottom of the bag loading device 12, this aids in the removal of the suture once loaded with autologous agents. The device 12 contains an exit point 16 as described in FIG. 2b. As with FIG. 1a the device 12 contains a connector port 18 such as a luer lock which is a docking point to attach the device 12 directly to a filter based blood concentrator or active loaded syringe.

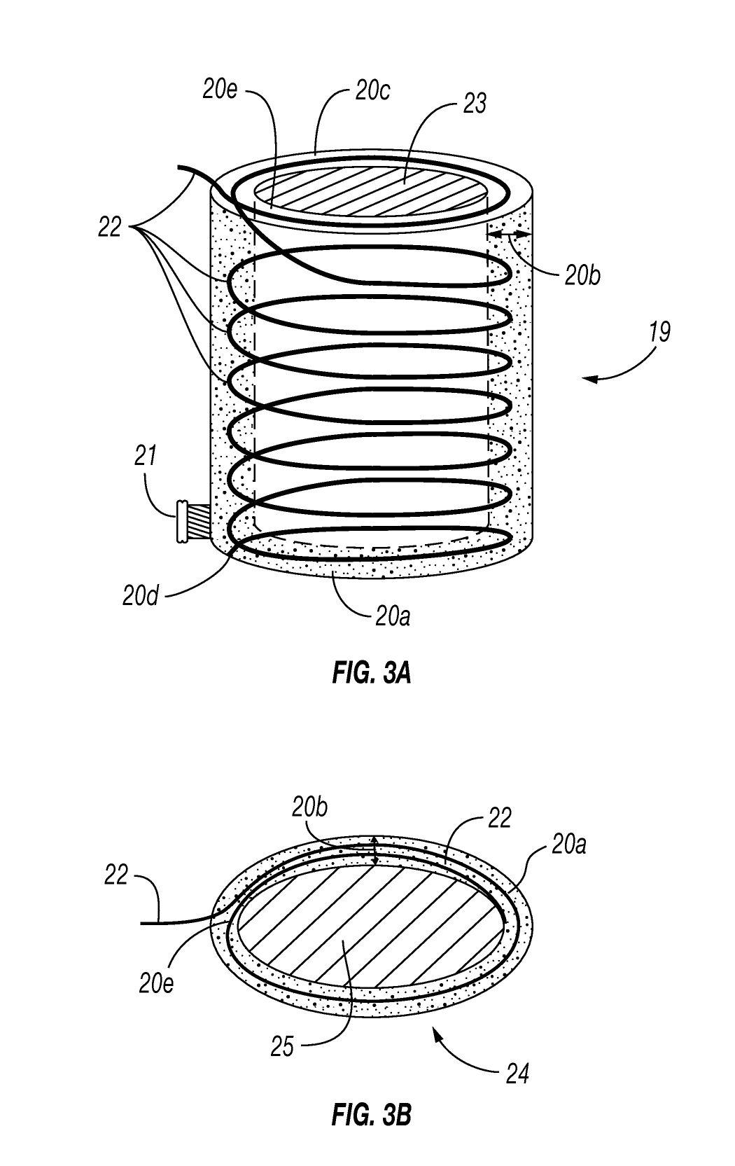

Various formats of medical device loading devices are envisaged. The loading device can be a thin double walled tubular shape (FIG. 3a). The loading device 19 is preferably made out of a solid material such as plastics, the tubular shape aids in the removal of the suture material 22 from the device. The loading device 19 has a double wall with hollow center 23 and a gap 20b between the walls which contains the suture material 22, and allows for the loading of autologous or active agents 20a into the device. The suture material 22 is wound around the device 19 during manufacture and is weakly attached at a point 20d within the device. This attachment point 20d breaks went significant force is applied to the suture 22 upon removal. The device 19 has a sealed top 20c with an exit point 20e for removal of the suture material 22. The exit point can be similar to then one described in FIG. 1b. As with previous embodiments the device 19 contains a connector port 21 such as a luer lock which is a docking point to attach the device 19 directly to a filter based blood concentrator or active loaded syringe. FIG. 3b illustrates a top view of the device shown in FIG. 3a. The top of the device 24 shows a hollow center 23 the wall gap 20b, autologous/active loaded agents 20a, a suture exit point 20 and a suture material 22.

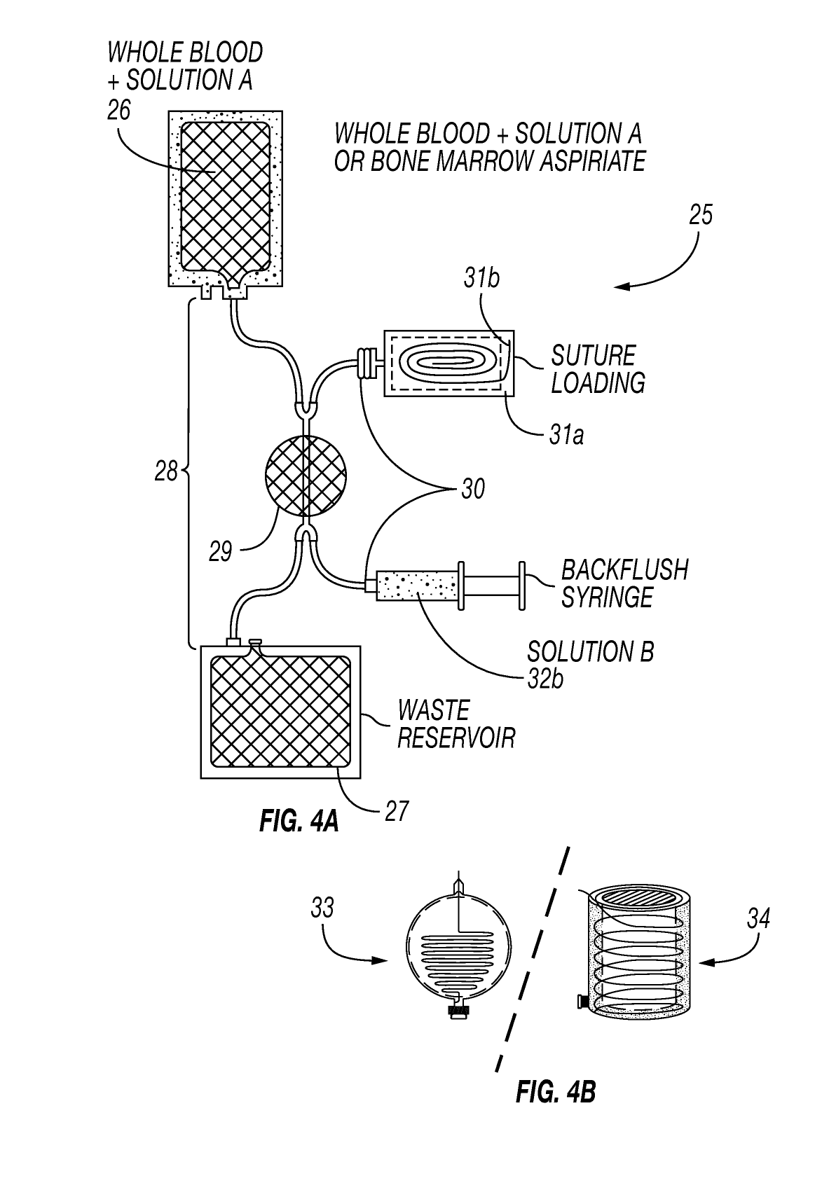

A schematic of the filter coupled with a suture loading device is shown in (FIG. 4), alternative loading devices 33, 34 are shown in FIG. 4b. The set-up 25 comprises of two standard 150-cc blood collection bags, a leukoreduction filter 29, and two ports 30 for both syringe and suture loading device attachment 31a. In using the device, whole blood was collected into blood bags containing 50-ml of Anticoagulant Citrate Dextrose, formula A (ACD-A). Subsequently, sixty ml aliquots were transferred into a 60-ml syringe.

The contents of the syringe were injected into a blood bag 26 comprising 10-ml of platelet capture solution A (water for injection). A platelet activation agent (e.g., calcium chloride solution or thrombin, or a combination thereof) may be added to the cell concentrate in order to activate platelets and induce coagulation. The addition of the platelet activator to the cell concentrate will result in a higher growth factor concentration (due to increased platelet activation) and better handling characteristics (due to coagulation).

The platelet activator should be added to the cell concentrate at an appropriate ratio to induce rapid platelet activation (less than 30 minutes) and coagulation. For example, a 1:10 activator:cell concentrate ratio is used when the activator is a CaCl.sub.2/thrombin (100 units/ml) solution.

The blood bag 26 comprising the diluted blood sample is attached to a platelet recovery filter 29 (Purecell P L, Pall Medical, Inc., Port Washington, N.Y.). The filtration height 28 (vertical distance between top of blood line in collection bag 26 and entry point into drain bag 27) was adjusted to 12.5 inches. After filtration was complete, the filter 29 was back flushed with a syringe filled with platelet recovery solution 32 B (5% saline solution) and 13-cc of air. The contents of the filter 29 were back flushed into a suture loading device 31a. The suture 31b within the device 31b wicks up the concentrated components. The suture can then be removed by the health professional and used in an appropriate indication.

What is claimed is:

1. A method for producing a fixation device comprising an autologous cell concentrate from a physiological solution, wherein the method comprises:
i) providing a physiological solution not previously subjected to centrifugation;
ii) subjecting the physiological solution to at least one filter to produce at least a first filter retentate and a first filter permeate solution, the first filter retentate comprising platelets, nucleated cells, or both, per unit volume greater than in the physiological solution, and wherein the first permeate solution comprises plasma and red blood cells;
iii) removing the first filter retentate from the filter to provide an autologous cell concentrate; and
iv) subjecting a fixation device to at least the first autologous cell concentrate.

2. The method of claim 1, wherein the fixation device is prepared intra-operatively.

3. The method of claim 1, wherein the first filter is a nucleated cell filtration device.

4. The method of claim 1, further comprising subjecting the first filter retentate to a second filtration.

5. The method of claim 4, wherein the second filtration utilizes a second filter comprising a hollow fiber filter.

6. The method of claim 5, wherein the first and/or second filters comprise a pore size between about 0.05 μm and about 5 μm.

7. The method of claim 5, wherein the first and/or second filters comprise a pore size between about 0.2 μm and about 0.5 μm.

8. The method of claim 1, further comprising combining an additional solution with the physiological solution, wherein the additional solution comprises bone marrow aspirate, blood, or a mixture thereof.

9. The method of claim 1, wherein the nucleated cells are at least one selected from the group consisting of stem cells, connective tissue progenitor cells, osteoprogenitor cells, or chondroprogenitor cells.

10. The method of claim 9, wherein the stem cells are mesenchymal stem cells, hematopoietic stem cells, or both.

11. The method of claim 1, wherein the fixation device is selected from at least one of the group consisting of sutures, tissue anchors, or bone anchors.

12. The method of claim 1, wherein the fixation device is a suture.

13. The method of claim 1, wherein providing the physiological solution comprises aspirating bone marrow from an individual into a first syringe to produce a bone marrow aspirate.

14. The method of claim 13, wherein the first syringe comprises an anti-coagulant, an isotonic solution, or both.

15. The method of claim 2, wherein the filtration and intra-operative loading is carried out in a single step.

16. A fixation device substantially coated with an autologous cell concentrate comprising:
i) a fixation device;
ii) at least one component of the fixation device substantially coated with an autologous cell concentrate, the autologous cell concentrate comprising substantially platelets, nucleated cells, or both, and substantially no plasma or red blood cells, wherein the autologous cell concentrate has been prepared by subjecting a physiological solution to a filter to produce a filter retentate and a permeate solution, the filter retentate comprising platelets, nucleated cells, or both per unit volume greater than in the physiological solution, and wherein the permeate solution comprises plasma and red blood cells.

17. The device of claim 16, wherein the fixation device is prepared intra-operatively.

18. The device of claim 16, wherein the filter is a nucleated cell filtration device.

19. The device of claim 18, wherein the filter comprises a pore size between about 0.05 μm and about 5 μm.

20. The device of claim 18, wherein the filter comprises a pore size between about 0.2 μm and about 0.5 μm.

21. The device of claim 16, the autologous cell concentrate further comprising bone marrow aspirate, blood, or a mixture thereof.

22. The device of claim 16, wherein the nucleated cells are at least one selected from the group consisting of stem cells, connective tissue progenitor cells, osteoprogenitor cells, or chondroprogenitor cells.

23. The device of claim 22, wherein the stem cells are mesenchymal stem cells, hematopoietic stem cells, or both.

24. The device of claim 16, wherein the fixation device is at least one selected from the group consisting of sutures, tissue anchors, or bone anchors.

25. The device of claim 16, wherein the fixation device is a suture.

26. The device of claim 16, wherein the at least one component of the fixation device that is coated is at least one strand of the suture.

27. An autologous cell concentrate coated suture comprising:
at least one strand of a suture substantially coated with an autologous cell concentrate comprising platelets, nucleated cells, or both.

28. The coated suture of claim 27, wherein the autologous cell concentrate comprises at least nucleated cells selected from the group consisting of stem cells, connective tissue progenitor cells, osteoprogenitor cells, or chondroprogenitor cells.

29. The coated suture of claim 27, wherein the autologous cell concentrate comprises stem cells.

30. The coated suture of claim 27, wherein the autologous cell concentrate comprises osteoprogenitor cells, chondroprogenitor cells, or both.
